(19) United States
(12) Patent Application Publication
(10) Pub. No.: US 2021/0183520 A1
(43) Pub. Date: Jun. 17, 2021

(54) MACHINE LEARNING BASED HEALTH OUTCOME RECOMMENDATION ENGINE

(71) Applicant: 7 Trinity Biotech Pte. Ltd., Singapore (SG)

(72) Inventor: Robert Alexandar Bates, Byron Bay (AU)

(21) Appl. No.: 16/936,328

(22) Filed: Jul. 22, 2020

Related U.S. Application Data
(60) Provisional application No. 62/948,658, filed on Dec. 16, 2019.

(51) Int. Cl. G16H 50/30; G06N 20/00; G06N 3/02; G16H 20/10; G16H 20/30; G16H 20/60; G16H 20/70; G16H 40/67; G16H 40/63

(52) U.S. Cl. CPC G16H 50/30; G06N 20/00; G06N 3/02; G16H 20/10; G16H 40/63; G16H 20/60; G16H 20/70; G16H 40/67; G16H 20/30

(57) ABSTRACT

A health outcome recommendation engine trains a machine-learned model using a training set of information that includes health characteristics, actions, environments, and health outcomes associated with training users. The health outcome recommendation engine applies the trained machine-learned model to health characteristics, actions, an environment, and health goals of a user. The machine-learned model identifies actions that, if taken by the user, increase a likelihood of the user achieving the health goals. The health outcome recommendation engine presents the actions to the user via an interface of a client device.

### CROSS REFERENCE TO RELATED APPLICATIONS

This application claims the benefit of U.S. Provisional Application No. 62/948,658, filed Dec. 16, 2019, which is incorporated by reference in its entirety.

### TECHNICAL FIELD

The disclosure generally relates to the field of health recommendations, and specifically to a machine learning based health outcome recommendation engine.

### BACKGROUND

A user may use applications on a client device to track fitness activities, diet, and vital signs, such as heart rate, among other health characteristics. While the user may be able to identify changes in and trends associated with these characteristics, conventional applications often fail to contextualize the user's health, and accordingly, do not provide feasible suggestions on how to improve the user's health.

### SUMMARY

A method for training and applying a machine-learned model configured to provide recommendations for achieving health outcomes is disclosed. The method includes accessing a training set of information comprising, for each of a plurality of training users, health characteristics of the training user, actions taken by the training user, an environment of the training user, a set of products used by the training user, and a set of health outcomes associated with the training user. The method includes training a machine-learned model based on the accessed training set of information. The trained machine-learned model is applied to a set of user information and a set of health goals received from a user. The user information describes one or more of health characteristics of the user, actions taken by the user, and an environment of the user. The machine-learned model is configured to identify actions that, if performed by the user, increase a likelihood that the user achieves the received set of health goals. The method subsequently includes modifying an interface displayed by a device of the user to include the identified actions. In some embodiments, a system and/or a non-transitory computer readable storage medium performs the steps described above.

### BRIEF DESCRIPTION OF DRAWINGS

The disclosed embodiments have other advantages and features which will be more readily apparent from the detailed description, the appended claims, and the accompanying figures (or drawings). A brief introduction of the figures is below.

FIG. 1 illustrates a system environment of a health outcome recommendation engine, in accordance with one or more embodiments.

FIG. 2 illustrates training and applying a machine-learned model configured to provide recommendations for achieving health outcomes, in accordance with one or more embodiments.

FIG. 3 illustrates an example process for providing a user with recommendations for achieving health outcomes, in accordance with one or more embodiments.

FIGS. 4A-C illustrate example user interfaces through which the user may interact with the health outcome recommendation engine, in accordance with one or more embodiments.

### DETAILED DESCRIPTION OF DRAWINGS

The Figures (FIGS.) and the following description relate to preferred embodiments by way of illustration only. It should be noted that from the following discussion, alternative embodiments of the structures and methods disclosed herein will be readily recognized as viable alternatives that may be employed without departing from the principles of what is claimed.

Reference will now be made in detail to several embodiments, examples of which are illustrated in the accompanying figures. It is noted that wherever practicable similar or like reference numbers may be used in the figures and may indicate similar or like functionality. The figures depict embodiments of the disclosed system (or method) for purposes of illustration only. One skilled in the art will readily recognize from the following description that alternative embodiments of the structures and methods illustrated herein may be employed without departing from the principles described herein.

### Overview

A user may track activities and health characteristics, such as diet, exercise, heart rate, and weight, via one or more applications on a client device. Use of these applications is often limited to viewing trends of and/or changes in the tracked activities and health characteristics. The method and system included herein describe a health outcome recommendation engine that takes in the user's health characteristics and actions, as well as an environment of the user, to recommend actions and/or products that would increase a likelihood of the user achieving a set of intended health goals. The health outcome recommendation engine selects recommendations for the user using a machine-learned model trained on a training set of information (such as other users' health information).

### System Environment

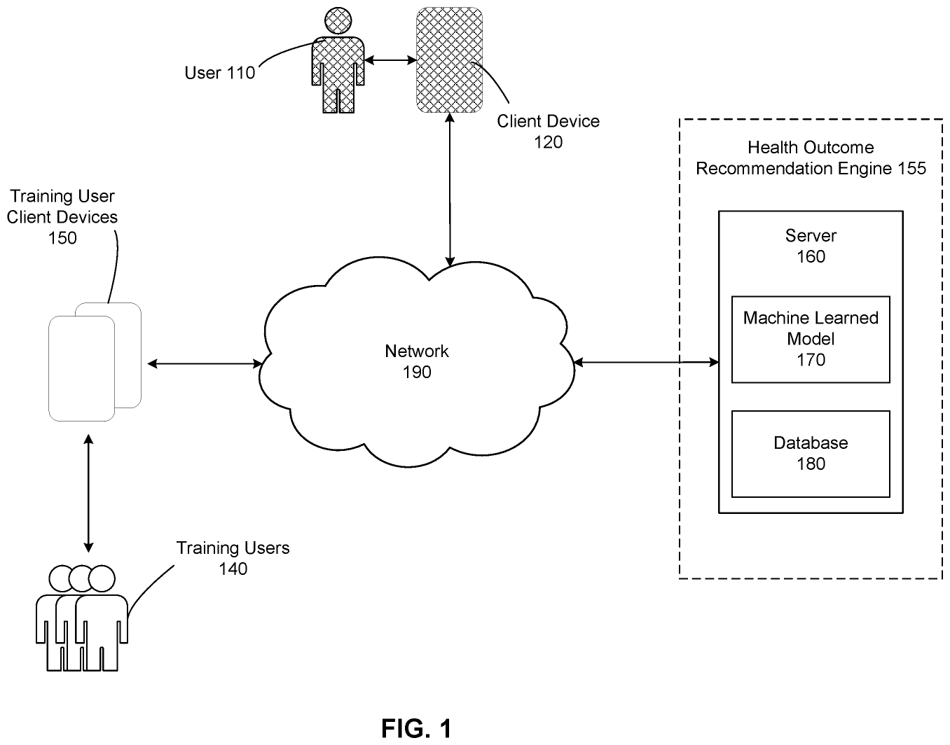

FIG. 1 illustrates a system environment of a health outcome recommendation engine, in accordance with one or more embodiments. The health outcome recommendation engine receives information about a user and provides the user with recommendations (such as product recommendations, action recommendations, and the like) that increase a likelihood of the user achieving the user's health goals. The system environment includes a user 110, a client device 120, a plurality of training users 140, a plurality of training user client devices 150, the health outcome recommendation engine 155, and a network 190.

The health outcome recommendation engine 155 provides recommendations to the user 110 to improve the likelihood that the user 110 will achieve the user's health goals. The health outcome recommendation engine 155 takes, as input, information about the user 110, such as health characteristics, actions, and environmental conditions around the user 110, and the user's health goals. In response to receiving the information about the user 110 and the user's health goals, the health outcome recommendation engine 155 generates recommendations for the user 110. The recommendations may include product suggestions (e.g., dietary supplements, topical serums, vitamins, etc.) as well as other action suggestions (e.g., meditation, physical activity, etc.).

The client device 120 couples the user 110 to the health outcome recommendation engine 155. The client device 120 is a computing device capable of transmitting and/or receiving data over the network 190. The client device 120 may be a conventional computer (e.g., a laptop or a desktop computer), a cellphone, or a similar device that communicates with the health outcome recommendation engine 155. In some embodiments, the client device 120 provides the health outcome recommendation engine 155 with the user's information and health goals. The client device 120 may be a device worn by the user 110 (e.g., a smart watch and/or a fitness tracker) that automatically collects data about the user's health characteristics, actions, and environmental conditions. In some embodiments, the user 110 inputs information about themselves to the health outcomes recommendation engine, via the client device 120. In some embodiments, another device, such as an Internet enabled blood sugar monitor, may couple to the client device 120 and provide information, in this case, the user's blood sugar, to the health outcome recommendation engine 155. In some embodiments, user health data can be provided by wearable fitness trackers or monitors, from health or monitoring applications running on a user's smart device, or from any other information source worn by, used by, or associated with a user. In some embodiments, multiple client devices 120 provide the health recommendation engine with information about the user. The client device 120 presents the recommendations to the user 110 as well, via a user interface displayed on the client device 120. In some embodiments, the health outcome recommendation engine 155 accesses a subset of the user's information from an external data source (e.g., environmental conditions at the user's location may be retrieved from a weather database and/or an air quality tracking system). In some embodiments, the health outcome recommendation engine 155 accesses a subset of the user's information from a social network profile of the user 110.

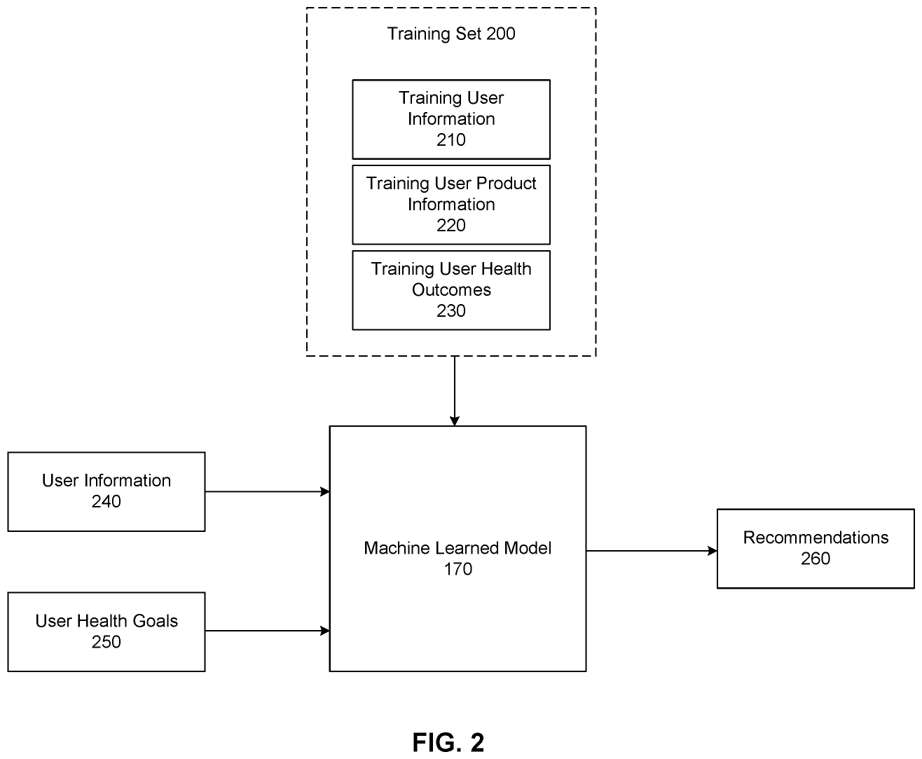

The health outcome recommendation engine 155 generates recommendations for the user 110 using a trained machine-learned model 170. The machine-learned model 170 is stored by the server 160 and trained using a training set of data including information about a plurality of training users 140. The training users 140 may be people other than the user 110 that use the health outcome recommendation engine. The training set includes, for each training user 140, health characteristics, actions, environmental conditions, one or more actions taken (such as health products used, exercises performed, and the like) and one or more health outcomes achieved by the training users 140. The training and application of the machine-learned model 170 is further described with respect to FIG. 2.

The training user client devices 150 provide information about the plurality of training users 140 to the server 160, over the network 190. The training user client devices 150 may be substantially similar to the client devices 120, and may be, for example, conventional computers or cellphones owned by each of the training users 140. In some embodiments, the training users 140 self-report a subset of their information to the health outcomes recommendation engine via their training user client device 150. In some embodiments, each training user's client device 150 automatically determines a subset of the training user's information (e.g., by receiving the data from a wearable fitness or health tracking device or from a health monitoring application running on the training user client device) and adds it to the training set. In some embodiments, the training user client devices 150 may automatically report health outcomes achieved by the training users 140, wherein the health outcomes are data received by the client devices 150, such as a heart rate, measure of blood pressure, etc. In some embodiments, the training users 140 self-report their health outcomes.

The server 160 stores and receives information from the client device 120 and the training user client devices 150. The server 160 hosts the machine-learned model 170 and the database 180. The server 160 may be located on a local or remote physical computer and/or may be located within a cloud-based computing system.

The database 180 stores information relevant to the recommendation engine. The database 180 stores information about the user 110, the user's health goals, and the training set comprising information about the plurality of training users 140.

The network 190 transmits data from the client device 120 and the training user client devices 150 to the server 160 and vice versa. The network 190 may be a local area and/or wide area network using wireless and/or wired communication systems, such as the Internet. In some embodiments, the network 190 transmits data over a single connection (e.g., a data component of a cellular signal, or WiFi, among others) and/or over multiple connections. The network 190 may include encryption capabilities to ensure the security of consumer data. For example, encryption technologies may include secure sockets layer (SSL), transport layer security (TLS), virtual private networks (VPNs), Internet Protocol security (IPsec), etc.

### Training and Application of Machine-Learned Model

FIG. 2 illustrates training and applying the machine-learned model 170, the machine-learned model 170 configured to provide recommendations for achieving health outcomes, in accordance with one or more embodiments. As described in FIG. 1, the machine-learned model 170 takes in information about a user (e.g., the user 110) and the user's health goals, and provides recommendations that will improve the likelihood that the user will achieve the user's health goals.

The machine-learned model 170 is trained using a set of training data (“training set 200”). The training set 200 includes training user information 210 (e.g., information about the training users 140), training user product information 220 (e.g., information about one or more products used by the training users 140), and training user health outcomes 230 (e.g., health outcomes of the training users 140). As described with respect to FIG. 1, the training user information 210, the training user product information 220, and/or the training user health outcomes 230 may be self-reported and/or automatically determined by client devices (e.g., the training user client devices 150) coupled to the health outcome recommendation engine 155. In some embodiments, the health outcome recommendation engine 155 incentivizes training users to provide the training user information 210, the training user product information 220, and/or the training user health outcomes 230 via gamification, rewards (such as social network status awards), and/or product offers. For example, a training user may receive an offer on a product if they provide information to the health outcome recommendation engine 155 for one month.

The training user information 210 includes information about a plurality of training users (e.g., the training users 140). For each training user, the training user information 210 includes at least one of health characteristics of the training user, actions taken by the training user, and an environment of the training user. Health characteristics may be measurements of and/or describe the training user's age, mental health, productivity, sleep, pollution, sexual and reproductive health, fertility, performance in sports, gastrointestinal microbiome, pain, cardiovascular health, pregnancy, post-natal health, immunity, disposition to and/or state of cancer, chronic inflammation, weight and/or obesity, eating disorders, substance use, access to healthcare, injury, vaccines, HIV and/or AIDS, nervous system, disposition to and/or history of stroke, lung disease, blood health (e.g., blood sugar, blood pressure), and non-communicable diseases (e.g., autoimmune disorders, heart disease, diabetes). The training user's actions may describe a lifestyle of the user, including a duration and quality of sleep, type and nature of physical activity (e.g. stretching, yoga, cardio, etc.), meditation, type and nature of employment, among others. The environment of the training user may be described by conditions of air quality (e.g., carbon dioxide concentrations, volatile organic compounds), temperature, ultraviolet radiation level, and humidity, among other parameters. In some embodiments, the training user information 210 includes data obtained via the training user's client device, such as a location of the training user via a GPS receiver and an itinerary of the training user via a calendar coupled to the training user's client device. For example, the health outcome recommendation engine 155 may determine conditions describing the environment around the training user based on training user's location via a GPS on the training user's client device. The health characteristics, actions, and environmental conditions associated with a training user may be recorded at set intervals over a period of time (e.g., every day for 5 weeks, every few hours, once every week).

The training user product information 220 includes information about one or more products used by the plurality of training users. In some embodiments, a product may correspond to a subset of the training user information 210. For example, the training user may consume a sleep aid (e.g., melatonin) to address poor sleep quality. The products included in the training user product information 220 may be nutritional supplements, vitamins, dietary supplements, neutraceutical supplements, topical creams and/or serums, lotions (such as lotions with sun protection factor or SPF), effervescent tablets and/or powders, essential oils, over the counter pharmaceuticals and/or medical devices, and diagnostic tools.

The training user health outcomes 230 describe health outcomes associated with the training user. Health outcomes may be associated with one or more health characteristics, actions, or environmental conditions of the training user. In some embodiments, health outcomes may be indirectly and/or directly related to the products used by the training user. Health outcomes may be positive or negative. Examples of positive health outcomes include reduced pain, reduced stress, greater sleep quality, reduced blood pressure, reduced inflammation, and improved energy levels, for example. In some embodiments, health outcomes may be negative side effects due to one or more products used by the user, such as anxiety, pain, nausea, dizziness, and skin conditions, among others. For example, a training user with a cardiovascular condition may react negatively to a particular product promoting anxiety relief.

The training users' information in the training set 200 may be considered a part of a positive training set or a negative training set. The positive training set includes health outcomes that positively impact the training user. For example, the training user information 210 training set may include health characteristics that indicate that a training user previously had trouble staying asleep for more than an hour at a time. When they used a sleep aid (e.g., melatonin), as indicated by the training user product information 220, the training user health outcomes 230 showed a significant increase in the training user's quality and duration of sleep. Thus, the health outcome associated with the sleep aid was positive for the training user.

The negative training set includes health outcomes that negatively impact the training user and/or have no impact on the training user. Continuing the above example, a different training user may react negatively to the sleep aid, resulting in a more disturbed sleep than usual. Thus, the health outcome associated with the sleep aid for a different training user may be negative. In another example, the health outcome may be neutral, neither positively nor negatively affecting the training user. For example, a training user with high blood sugar and decent sleep patterns may not experience any effects from consuming the sleep aid. Accordingly, the training set 200 provides the machine-learned model 170 with information about a training user, information describing the training user, products used by the training user, and associated health outcomes. The training set 200 may be categorized into a positive training set and a negative training set.

The health outcome recommendation engine 155 uses supervised or unsupervised machine learning to train the machine-learned model 170 using the positive and/or negative training sets of the training set 200 to enable the machine-learned model to identify correlations and relationships between health outcomes (e.g., an ability or likelihood of a user to meet a health goal) and user information, product use information, action information, and environmental information. Different machine learning techniques may be used in various embodiments, such as linear support vector machine (linear SVM), boosting for other algorithms (e.g., AdaBoost), neural networks, logistic regression, naive Bayes, memory-based learning, random forests, bagged trees, decision trees, boosted trees, or boosted stumps.

In one embodiment, the machine-learned model 170 creates a matrix, based on the training set 200, describing each training user's overall state of health. The matrix tracks the user information 240 across time, where each row of the matrix corresponds to a health characteristic, action, or environmental condition, and each column corresponds to a point in time. The machine-learned model 170 creates vectors for each product of the training user product information 220, the vectors identifying the benefits that the product has demonstrated for each health characteristic, action, or environmental condition. Accordingly, the trained machine-learned model 170 identifies relationships between the training user information 210, the training user product information 220 and the associated training user health outcomes 230 and uses the matrix and generated product vectors to provide recommendations to users. It should be noted that the machine-learned model can be trained using vectors corresponding to actions taken by training users other than product usage according to the principles described herein.

The trained machine-learned model 170, when applied to another user's (e.g., the user 110) information 240 and health goals 250, outputs recommendations 260 that increase the likelihood of the user achieving the user's health goals. As described with respect to FIG. 1, the user's information 240 and health goals 250 may be reported to the health outcome recommendation engine 155 by the user via a client device (e.g., the client device 120) and/or automatically determined by the client device. The user may be incentivized to provide the user information 240 and the user health goals 250 via gamification, rewards, and/or product offers.

The user information 240 includes the user's health characteristics, actions, and environment, and can include examples substantially similar to those described with respect to the training user. The user health goals 250 may be associated with the user information 240. For example, the user health goals 250 may include the user's aim to sleep at least 8 hours per night, exercise 5 times per week, lose at least 5 pounds in 2 months, among others. In some embodiments, the user health goals 250 are associated generally with a health characteristic, action, or environment. For example, the user may set a health goal to reduce a level of stress the user is experiencing. In some embodiments, the user health goals 250 may be generalized across a demographic due to research data. For example, research may show that 8 hours of sleep is necessary for people under the age of 20. Accordingly, the health outcome recommendation engine 155 may automatically set at least one user health goal 250 to achieve 8 hours of sleep each night.

Based on the input user information 240, the trained machine-learned model 170 may generate a matrix that describes the user's overall state of health. Similar to the matrix built from the training set 200, the matrix of the user's state of health includes rows corresponding to a health characteristic, action, or environmental condition, and each column corresponds to a point in time. The machine-learned model 170 calculates a dot product of each product vector and the user matrix; the resultant dot product with the highest value for each health characteristic, action or environmental condition indicates which product has the highest likelihood of improving the respective characteristic. In some embodiments, the machine-learned model 170 recommends the product with the biggest overall impact on the user's health (e.g., the maximum resultant dot product). Alternatively, the user information 240 may be formatted into any suitable format used required by the machine-learned model 170 (such as the format of the training information used to train the machine-learned model).

The recommendations 260 include suggested products and other actions that will help the user achieve the user's health goals 250. Recommended products may be similar to the products used by the training users, such as those included in the training user product information. Recommended actions include suggested meditation sessions, social activities, physical activities, stretches, reminders to drink water, and food suggestions, among others. For example, in response to receiving user information 240 with health characteristics that indicate that the user has poor mental health and user health goals 250 of wanting to feel less anxiety, the machine-learned model 170 may provide recommendations 260 prompting the user to go for a walk in a nearby park. In another example, the user information 240 indicates that the user has a poor gastrointestinal microbiome, and the user health goals 250 indicate that the user wants to be able to eat without a gastrointestinal reaction. The machine-learned model 170 outputs recommendations 260 for dietary supplements, such as probiotics, that will assist with achieving the user's health goals 250.

In some embodiments, the machine-learned model presents recommendations 260 based on the user information 240, independent of the user health goals 250. For example, in response to determining that air pollution at the user's location (e.g., determined via the user's client device) is at unhealthy levels, the machine-learned model's recommendations 260 may include products associated with reducing asthma.

The recommendations 260 are displayed on the client device, in some embodiments, by modifying a display of the client device. In some embodiments, the client device notifies the user of recommendations 260.

### Process for Presenting Recommendations

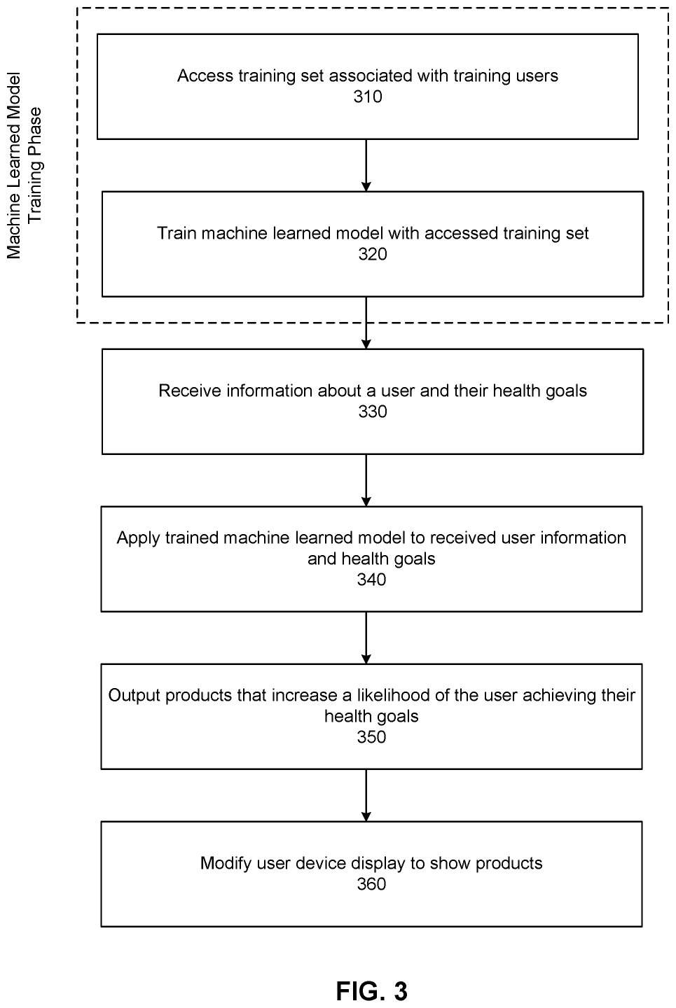

FIG. 3 illustrates an example process for providing a user with recommendations for achieving health outcomes, in accordance with one or more embodiments. A health outcome recommendation engine accesses 310 a training set (e.g., the training set 200) associated with a plurality of training users (e.g., the training users 140). For each training user, the training set includes information about health characteristics, actions, and environmental conditions, as well as any products used, and associated health outcomes.

The health outcome recommendation engine trains 320 a machine-learned model (e.g., the machine-learned model 170) with the training set. The machine-learned model determines relationships between the actions taken and products used by training users and their health outcomes.

The health outcome recommendation engine receives 330 information about a user (e.g., the user information 240) and the user's health goals (e.g., the user health goals 250). The user (e.g., the user 110) is distinct from the training users. The user information can include health characteristics, actions, and environmental conditions tracked over a period of time.

The health outcome recommendation engine applies 340 the trained machine-learned model to the received user information and health goals. The trained machine-learned model identifies products and/or actions that increase a likelihood of the user achieving the user's health goals, and outputs 350 the identified products and/or actions as recommendations.

The health outcome recommendation engine 360 modifies a display of a client device of the user (e.g., the client device 120) to include the recommended products and/or actions that will aid the user in achieving the user's health goals.

In some embodiments, the user presents feedback to the health outcome recommendation engine as to whether the recommended products and/or actions helped achieve the user's health goals. The presented feedback is added to the training set to improve the machine-learned model's recommendations, for instance by retraining the machine-learned model.

### Example User Interface

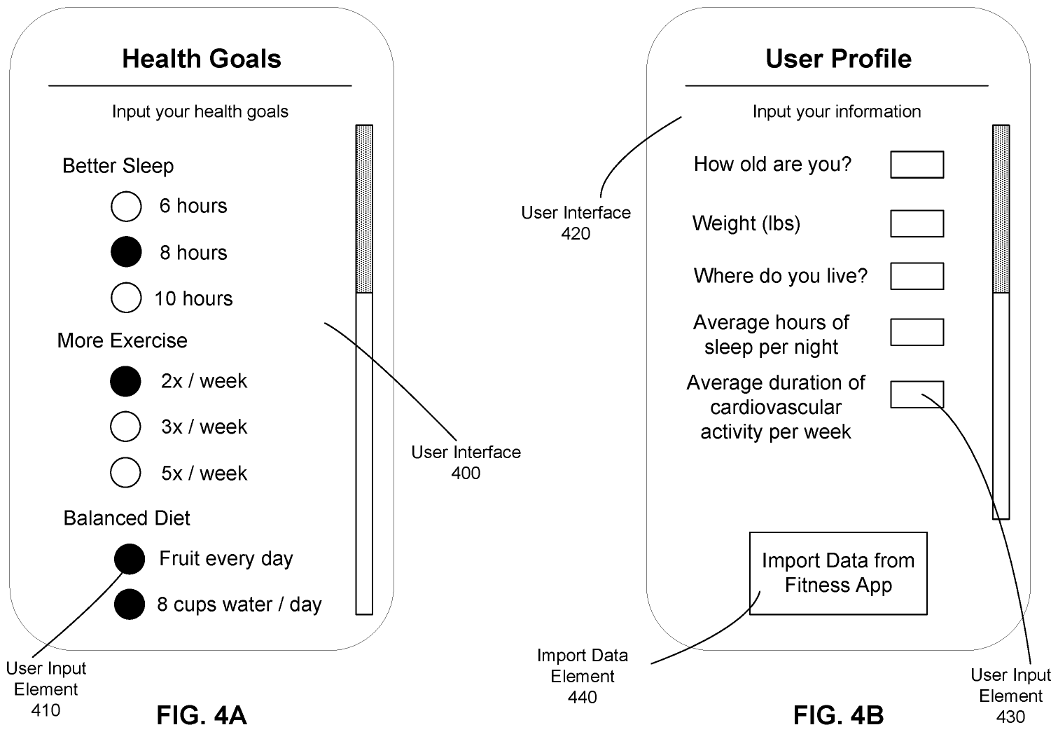

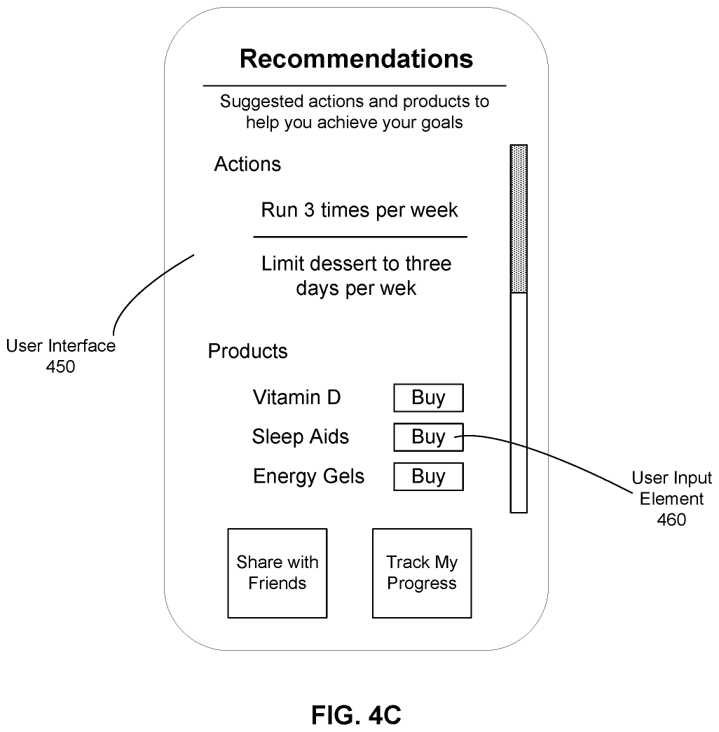

FIGS. 4A-C illustrate an example user interfaces through which the user may interact with the health outcome recommendation engine, in accordance with one or more embodiments. In some embodiments, the user accesses the health outcome recommendation engine via an application executed on a client device (e.g., the client device 120). The client device displays the user interfaces of the health outcome recommendation engine, and enables the user to provide input to and/or interact with the health outcome recommendation engine.

In FIG. 4A, the user interface 400 enables the user to input health goals via a plurality of user input elements 410. As described above, health goals include, for example, a desire for better sleep, more exercise, and/or a balanced diet. In some embodiments, the user interface 400 allows the user to input custom health goals. The user may opt to automatically import health goals into the health outcome recommendation engine from, for example, another application on the client device or from a wearable fitness device (such as a smart watch) worn by the user. In some embodiments, the health outcome recommendation engine may contact a medical professional associated with the user to determine the user's health goals. In other embodiments, the medical professional may provide input, via the user input elements 410, on the user's health goals.

In FIG. 4B, the user interface 420 enables the user to input information about the user's health characteristics, activities, and environment via the user input elements 430. In some embodiments, the user interface 420 includes an import data element 440. When interacted with, the import data element 440 extracts the user's information from another application hosted and/or executed on the client device (e.g., a fitness tracking application, a social networking application). In some embodiments, the health outcome recommendation engine automatically receives the user's information from the client device and/or another application on the client device.

In FIG. 4C, the user interface 450 presents recommendations to the user on actions and/or products that increase a likelihood that the user achieves the input health goals. In FIG. 4C, the user interface 450 includes user input elements 460 that allow the user to buy the suggested products, share suggested actions and/or products with friends (e.g., via social media), and track progress over time (e.g., by displaying one or more interfaces or visualizations of a user's progress in achieving one or more health goals). In some embodiments, the user may provide feedback to the health outcome recommendation engine on whether the suggested actions and/or products helped achieve the health goals and/or how likely the user is to follow through on the recommendations, and the user interface 450 can, in response, display new or additional recommendations identified by the machine-learned model to increase the likelihood that the user achieves the identified health goals.

### Additional Configuration Considerations

The foregoing description of the embodiments has been presented for the purpose of illustration; it is not intended to be exhaustive or to limit the patent rights to the precise forms disclosed. Persons skilled in the relevant art can appreciate that many modifications and variations are possible in light of the above disclosure.

Some portions of this description describe the embodiments in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like.

Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. In one embodiment, a software module is implemented with a computer program product comprising a computer-readable medium containing computer program code, which can be executed by a computer processor for performing any or all of the steps, operations, or processes described.

Embodiments may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a non-transitory, tangible computer readable storage medium, or any type of media suitable for storing electronic instructions, which may be coupled to a computer system bus. Furthermore, any computing systems referred to in the specification may include a single processor or may be architectures employing multiple processor designs for increased computing capability.

Embodiments may also relate to a product that is produced by a computing process described herein. Such a product may comprise information resulting from a computing process, where the information is stored on a non-transitory, tangible computer readable storage medium and may include any embodiment of a computer program product or other data combination described herein.

Finally, the language used in the specification has been principally selected for readability and instructional purposes, and it may not have been selected to delineate or circumscribe the patent rights. It is therefore intended that the scope of the patent rights be limited not by this detailed description, but rather by any claims that issue on an application based hereon. Accordingly, the disclosure of the embodiments is intended to be illustrative, but not limiting, of the scope of the patent rights, which is set forth in the following claims.

What is claimed is:

1. A method comprising:
accessing a training set of information comprising, for each of a plurality of training users, health characteristics of the training user, actions taken by the training user, an environment of the training user, a set of products used by the training user, and a set of health outcomes associated with the training user;
training a machine-learned model based on the accessed training set of information, the machine-learned model configured to identify correlations between user information and actions that, if taken, increase a likelihood that a user achieves one or more health goals;
receiving, from a user, user information describing one or more of: health characteristics of the user, actions taken by the user, and an environment of the user;
receiving, from the user, a set of health goals;
applying the machine-learned model to the received user information and the set of health goals, the machine-learned model configured to identify one or more actions that, if taken by the user, increase a likelihood that the user achieves the received set of health goals; and
modifying an interface displayed by a device of the user to include the identified one or more actions.

2. The method of claim 1, wherein the machine-learned model identifies relationships between products used by the plurality of training users, the health outcomes associated with the plurality of training users, and one of: the health characteristics of the plurality of training users, the actions taken by the plurality of training users, and the environments of the plurality of training users.

3. The method of claim 1, wherein the identified one or more actions include using one or more medical products comprising one or more of: dietary supplements, topical lotions, vitamins, diagnostic tools, and medical devices.

4. The method of claim 3, wherein the one or more medical products comprise one or more of: dietary supplements, topical lotions, vitamins, diagnostic tools, and medical devices.

5. The method of claim 1, wherein the one or more actions comprise one or more of: physical activity, social activity, and relaxation.

6. The method of claim 1, further comprising:
receiving input from the user indicating whether the identified one or more actions improved the likelihood of the user achieving the received set of health goals;
updating the training set of information based on the received input from the user; and
retraining the machine-learned model based on the updated training set of information.

7. The method of claim 1, further comprising:
responsive to receiving, via a client device, health characteristics of a training user, actions taken by the training user, an environment of the training user, a set of products used by the training user, and a set of health outcomes associated with the training user, providing the training user with a reward or an offer on the identified one or more products.

8. The method of claim 1, wherein the user information describing one or more of the health characteristics of the user, actions taken by the user, and the environment of the user are received periodically.

9. The method of claim 8, wherein the machine-learned model identifies the one or more actions by:
generating a matrix describing the user's state of health, the matrix comprising:
rows that correspond to health characteristics of the user, actions taken by the user, and an environment of the user; and
columns that correspond to each interval of time.

10. The method of claim 9, wherein the machine-learned model generates a vector for each of the identified one or more actions, the vector comprising a set of entries, each entry representative of a benefit of the action corresponding to each row.

11. The method of claim 10, further comprising:
responsive to determining a dot product of the vector for each of the identified one or more actions and the user matrix, identifying at least one action associated with a greatest dot product to recommend.

12. A non-transitory computer readable storage medium comprising computer executable code that when executed by one or more processors causes the one or more processors to perform operations comprising:
accessing a training set of information comprising, for each of a plurality of training users, health characteristics of the training user, actions taken by the training user, an environment of the training user, a set of products used by the training user, and a set of health outcomes associated with the training user;
training a machine-learned model based on the accessed training set of information, the machine-learned model configured to identify correlations between user information and actions that, if taken, increase a likelihood that a user achieves one or more health goals;
receiving, from a user, user information describing one or more of: health characteristics of the user, actions taken by the user, and an environment of the user;
receiving, from the user, a set of health goals;
applying the machine-learned model to the received user information and the set of health goals, the machine-learned model configured to identify one or more actions that, if taken by the user, increase a likelihood that the user achieves the received set of health goals; and
modifying an interface displayed by a device of the user to include the identified one or more actions.

13. The non-transitory computer readable storage medium of claim 12, wherein the machine-learned model identifies relationships between products used by the plurality of training users, the health outcomes associated with the plurality of training users, and one of: the health characteristics of the plurality of training users, the actions taken by the plurality of training users, and the environments of the plurality of training users.

14. The non-transitory computer readable storage medium of claim 12, wherein the identified one or more actions include using one or more medical products comprising one or more of: dietary supplements, topical lotions, vitamins, diagnostic tools, and medical devices.

15. The non-transitory computer readable storage medium of claim 12, wherein the operations further comprise:
receiving input from the user indicating whether the identified one or more actions improved the likelihood of the user achieving the received set of health goals;
updating the training set of information based on the received input from the user; and
retraining the machine-learned model based on the updated training set of information.

16. The non-transitory computer readable storage medium of claim 12, wherein the operations further comprise responsive to receiving, via a client device, health characteristics of a training user, actions taken by the training user, an environment of the training user, a set of products used by the training user, and a set of health outcomes associated with the training user, providing the training user with a reward or an offer on the identified one or more products.

17. The non-transitory computer readable storage medium of claim 12, wherein the user information describing one or more of the health characteristics of the user, actions taken by the user, and the environment of the user are received periodically.

18. A computer system comprising:
one or more computer processors; and
a non-transitory computer readable storage medium comprising computer executable code that when executed by the one or more processors causes the one or more processors to perform operations comprising:
accessing a training set of information comprising, for each of a plurality of training users, health characteristics of the training user, actions taken by the training user, an environment of the training user, a set of products used by the training user, and a set of health outcomes associated with the training user;
training a machine-learned model based on the accessed training set of information, the machine-learned model configured to identify correlations between user information and actions that, if taken, increase a likelihood that a user achieves one or more health goals;
receiving, from a user, user information describing one or more of: health characteristics of the user, actions taken by the user, and an environment of the user;
receiving, from the user, a set of health goals;
applying the machine-learned model to the received user information and the set of health goals, the machine-learned model configured to identify one or more actions that, if taken by the user, increase a likelihood that the user achieves the received set of health goals; and
modifying an interface displayed by a device of the user to include the identified one or more actions.

19. The computer system of claim 18, wherein the machine-learned model identifies the one or more actions by:
generating a matrix describing the user's state of health, the matrix comprising:
rows that correspond to health characteristics of the user, actions taken by the user, and an environment of the user; and
columns that correspond to each interval of time.

20. The computer system of claim 19, wherein the machine-learned model generates a vector for each of the identified one or more actions, the vector comprising a set of entries, each entry representative of a benefit of the action corresponding to each row.
